(19) United States
(12) Patent Application Publication
(10) Pub. No.: US 2007/0185008 A1
(43) Pub. Date: Aug. 9, 2007

(54) Stereocomplex hydrogels with tunable degradation times

(76) Inventors: Wilhelmus Everhardus Hennink, Wadddinxveen (NL); Cornelis Franciscus van Nostrum, Vlijmen (NL)

Correspondence Address: MORRISON & FOERSTER LLP, 12531 HIGH BLUFF DRIVE, SUITE 100, SAN DIEGO, CA, 92130-2040, US

(21) Appl. No.: 10/581,369

(22) PCT Filed: Dec. 3, 2004
(86) PCT No.: PCT/NL04/00845

§ 371 Date: Apr. 10, 2007

(30) Foreign Application Priority Data
Dec. 4, 2003 (EP) 03078852.5

(51) Int. Cl. A61K 47/42; C08L 1/02; C08L 1/08; C08L 5/02

(52) U.S. Cl. 514/2; 514/772.1; 525/451; 525/50; 525/509; 525/54.23; 525/54.26

(57) ABSTRACT

The present invention relates to stereocomplex hydrogels for drug delivery and tissue engineering. The hydrogels comprise block or graft polymers with at least one hydrophilic region and at least two enantiomerically enriched degradable regions, which may represent grafts or terminal blocks. In the hydrogels, degradable regions of opposite chirality form racemic crystallites, leading to the physical crosslinking of the polymers. Furthermore, the significance of the terminal groups of the degradable blocks for the degradability of the hydrogel is disclosed. Hydrogels from polymers whose degradable regions are characterised by the absence of terminal hydroxyl groups are shown to be particularly stable, having long lifetimes and a high potential for sustained drug release over extended periods such as weeks or months. In other aspects, the invention provides methods for the preparation of hydrogel compositions, kits from which the hydrogels can be prepared, and uses of the hydrogels.

### BACKGROUND

The present invention relates to physical hydrogel compositions, and particularly to stereocomplex hydrogels, especially for drug delivery and tissue engineering. The compositions of the invention represent hydrated, three-dimensional polymeric networks in which polymer chains are crosslinked with each other primarily by non-covalent links. More specifically, the invention relates to stereocomplex hydrogels, in which the polymers have regions of opposite chirality capable of forming stereocomplexes, which are racemic crystallites. In a further aspect, the invention relates to methods for the preparation of such stereocomplex hydrogel compositions, and the use thereof. Furthermore, kits are provided from which stereocomplex hydrogel compositions can be prepared.

Biodegradable hydrogels are an important class of materials for tissue engineering and for the controlled release of pharmaceutically active compounds such as therapeutic proteins. Hydrogels are three-dimensional polymeric networks made by chemical or physical crosslinking of hydrophilic polymers (Hennink W E and Van Nostrum C F. Novel crosslinking methods to design hydrogels. Adv. Drug Del. Rev. 54, 13-36, 2002). In chemically crosslinked gels, the polymers are connected primarily by covalent bonds. In physically crosslinked gels, the network is formed by physical or physicochemical interactions between different polymer chains. In recent years, there has been an increasing interest in physically crosslinked gels, especially in those gel compositions in which gel formation occurs under mild conditions and in the absence of organic solvents. The main reason for this interest is that the use of crosslinking reagents and organic solvents, which tend to have detrimental effects on bioactive proteins which are often incorporated into these gels as active substances, can be avoided in the preparation of drug delivery systems or tissue engineering matrices based on such gels. These agents and solvents can not only affect the active substances to be entrapped, but they are often relatively toxic compounds whose residuals have to be removed carefully from the gels before these can be used.

A great variety of methods have been applied to create physically crosslinked gels which use ionic, hydrophobic and hydrogen bond interactions. A more recent approach is the formation of crystalline domains in polymeric networks, i.e. crystallites, which are insoluble in water at physiological conditions. This has been described for linear polymer chains with multiple hydroxyl groups, such as poly(vinylalcohol).

Furthermore, crystallites can be formed from polymers composed of optically active, chiral, monomeric units. If polymer regions with opposite chirality are mixed, these regions can associate and form racemic crystalline domains, which are referred to as stereocomplexes. Stereocomplex hydrogels can, for instance, be formed by mixing enantiomerically enriched polymers of opposite chirality. Alternatively, they can be formed from only one polymeric species having regions of opposite chirality.

For example, WO 00/48576 discloses stereocomplex hydrogels prepared from a mixture of polymers having complementary, i.e. opposite, chirality. The chiral regions are primarily composed of units derived from lactic acid. In particular, graft polymers are described in which oligo(lactate) grafts represent the chiral regions.

De Jong et al. (J. Controlled Release 72, 47-56, 2001) also describe biodegradable hydrogels based on stereocomplex formation between D- and L-lactic acid oligomers grafted to dextran backbones. Lim et al. (Macromol. Rapid Commun. 21, 464-471, 2000) developed hydrogels from two enantiomeric amphiphilic graft copolymers having backbones of poly(2-hydroxyethyl methacrylate) and side chains of oligo(D-lactide) or oligo(L-lactide), respectively. Also in these hydrogels, stereocomplex formation occurs between the side chains of opposite chirality.

These stereocomplex hydrogels, which have been suggested for drug delivery applications, are biodegradable by virtue of their hydrolysable oligomeric lactide side chains, which occurs at a moderate rate under physiological conditions. Other degradable structures may also be present in the polymers. For example, the linking groups between the polymer backbone and the grafts may contribute to the overall degradability of the hydrogel.

Many of these known stereocomplex gels show a hydrolysis that is not slow enough to provide sufficient gel stability and drug retention over several weeks or months. Such slower hydrolysis behaviour would however be desirable for many, if not most, presently envisioned controlled release applications.

Thus there is a need for stereocomplex hydrogels which are physically stable for longer time periods than the presently known stereocomplex gels, and which, hence, degrade very slowly under physiological conditions, and which are potentially capable of releasing incorporated active ingredients, such as therapeutic proteins, over several weeks or even months.

It is an object of the inventions to provide such hydrogels with improved chemical and physical stability, and to provide compositions comprising such hydrogels. Another object is to provide uses for such hydrogels and methods for their preparation. Further objects of the invention will become clear on the basis of the following description.

### SUMMARY OF THE INVENTION

According to the invention, stereocomplex hydrogel compositions are provided which comprise a mixture of a first and a second polymer. Both the first and the second polymer individually have at least one hydrophilic region and at least two oligomeric degradable regions which are hydrolysable under physiological conditions. These at least two degradable regions comprise enantiomerically enriched chiral monomeric units. At least one of the degradable regions of the first polymer and and at least one of the degradable regions of the second polymer have predominantly opposite chirality. The invention is further characterised in that at least some of the degradable regions present in the composition have no free terminal hydroxyl groups, i.e. at least some polymer molecules representing either the first or the second polymer comprise a degradable region without free terminal hydroxyl groups.

Preferably, the hydrogels comprise block or graft polymers with at least one hydrophilic region and at least two enantiomerically enriched degradable regions, which may represent grafts or terminal blocks.

In the hydrogels, degradable regions of opposite chirality form racemic crystallites, leading to the physical crosslinking of the polymers. Furthermore, the significance of the terminal groups of the degradable blocks for the degradability of the hydrogel is disclosed. Hydrogels from polymers whose degradable regions are characterised by the absence of terminal hydroxyl groups are shown to be particularly stable, having long lifetimes and a high potential for sustained drug release over extended periods such as weeks or months.

The first and the second polymer are preferably different from each other, the difference being the chirality of their degradable regions. In other words, each of the two polymers comprises only one of the two chiral species in its degradable regions. Optionally, however, the first and the second polymer can be identical if each polymer molecule comprises regions of both chiralities.

As noted, the polymers preferably represent graft polymers in which the hydrophilic region is the backbone and the degradable regions are grafts, side chains of the polymers. Alternatively, the polymers may represent block polymers, such as ABA block polymers, in which at least the terminal blocks of the polymer chain are formed by degradable regions, whereas the hydrophilic region is the block, or one of the blocks, positioned in between the terminal blocks.

In another aspect, methods are provided for the preparation of the hydrogel compositions of the invention. The methods comprise a step of combining a first and a second component in the presence of water and, optionally, other excipients. The first component comprises at least one of the first and the second polymer as defined in claim 1. If the first and the second polymer are different from each other, i.e. if they comprise degradable regions with opposite chirality, it is preferred that the first component comprises the first polymer and the second component comprises the second polymer.

In a further aspect, the use of such stereocomplex hydrogels and hydrogel compositions in drug delivery and tissue engineering is provided, particularly as components of injectable or implantable pharmaceutical formulations providing controlled release of active compounds such as therapeutic proteins.

In yet another aspect, kits are provided from which the stereocomplex hydrogel compositions of the invention can be prepared.

### DETAILED DESCRIPTION OF THE INVENTION

The invention provides novel and improved stereocomplex hydrogels and hydrogel compositions for drug delivery and tissue engineering applications. According to a first aspect, a stereocomplex hydrogel composition is provided which comprises a mixture of first and second polymers. Both the first and the second polymer individually have at least one hydrophilic region and at least two oligomeric degradable regions which are hydrolysable under physiological conditions. These at least two degradable regions comprise enantiomerically enriched chiral monomeric units. At least one of the degradable regions of the first polymer and one of the degradable regions of the second polymer have predominantly opposite chirality. The invention is further characterised in that at least some of the degradable regions present in the composition have no free terminal hydroxyl groups, i.e. at least some polymer molecules representing either the first or the second polymer comprise a degradable region without free terminal hydroxyl groups.

As used herein, hydrogels are water-swollen, three-dimensional polymeric networks in which polymer chains are physically or chemically crosslinked. Depending on the nature of crosslinks, a hydrogel may be termed a chemical or a physical hydrogel. At room or body temperature, hydrogels are basically insoluble in water. Hydrogel compositions are compositions comprising a hydrogel and, optionally, further constituents.

Stereocomplex hydrogels are “physical” hydrogels in which stereocomplexes are present which complexes function as crosslinks between the participating polymer molecules. Stereocomplexes are racemic crystallites, or crystalline regions, formed by structures such as polymeric or oligomeric regions (such as grafts or blocks) of opposite chirality. In addition to stereocomplexes, other types of crosslinks may be present in a stereocomplex hydrogel and contribute to its stability.

The regions of opposite chirality can be present in polymers which are herein referred to as the first and the second polymer. The first and the second polymer are preferably different from each other, the differences being at least the chirality of their degradable regions. This means that each of the two polymers comprises only or predominantly one of the two chiral species in its degradable regions.

Optionally, however, the first and the second polymer may be identical. This is possible if each polymer molecule comprises regions of both chiralities.

The stereocomplexes are formed from regions which are chirally complementary, i.e. which have predominantly opposite chirality. This means that these regions must be predominantly comprised of chiral monomeric units, and that they must be enantiomerically enriched. As used herein, “enantiomerically enriched” refers to structures whose chiral monomeric units are either selected from only one enantiomer, or in which the content of one enantiomer is significantly higher than the content of the other enantiomer. For example, regions comprised of lactate units are enantiomerically enriched of they contain exclusively (L)- or (D)-lactate units, but also if they contain both enantiomers, but in such a ratio that a stereocomplex formation is still possible. Generally, enantiomeric enriched in one enantiomer means that said enantiomer is present relative to the other enantiomer in a ratio of at least about 8:2, and preferably at least 9:1. In other words, the term “enantiomerically enriched” also includes structures which are not enantiomerically pure.

In analogy, regions may also be termed chirally complementary, or referred to as having opposite chirality, if they are not enantiomerically pure. Furthermore, they may comprise a limited number of units which are not chiral at all. For example, the terms are used to include oligomeric (L)- or (D)-lactate regions which also contain some glycolate, caprolactone, or propriolactone units.

The degradable regions may represent grafts or blocks of the first and/or of the second polymer. More preferably, the polymers on which the hydrogel compositions of the invention are based represent graft polymers in which the hydrophilic region is the backbone and the degradable regions are grafts, or side chains of the polymers. In the terminology of the invention, graft polymers are understood as a polymer with one or more species of block connected to the main chain, or backbone, as side-chains, and wherein these side-chains have constitutional or configurational features that differ from those in the backbone. A side chain, also called branch or pendant chain, is an offshoot from the main chain.

Alternatively, the polymers on which the hydrogels are based may represent block polymers, such as ABA block polymers, in which at least the terminal blocks of the polymer chain are formed by degradable regions, whereas the hydrophilic region is the block, or one of the blocks, positioned in between the terminal blocks. Block polymers are generally defined as polymers composed of blocks which are arranged in a linear sequence. A block of a block polymer has constitutional or configurational features that make the block different from the adjacent blocks. Optionally, such block polymer may also comprise grafts, and thus represent a block polymer and a graft polymer at the same time.

As mentioned above, the hydrophilic region of the polymer or polymers on which the hydrogels are based may be represented by a non-terminal block if the respective polymer is a block polymer, or by the backbone if the respective polymer is a graft polymer. Hydrophilic means that the region is predominantly composed of monomeric units whose homopolymers are water soluble or water-dispersible. Alternatively, if the hydrophilic region is a backbone comprised of different monomeric units either randomly or as blocks, the main chain as a whole (without side chains) is water soluble or water-dispersible.

For the avoidance of misunderstandings, the hydrophilic region as defined herein may also be degradable to some extent. Likewise, the degradable regions possess some degree of hydrophilicity, or comprise substituents which are hydrophilic. According to the invention, however, the terms “hydrophilic region” and “degradable region” never specify one and the same region.

In a preferred embodiment, the polymers from which the hydrogel of the invention is composed are preferably graft polymers having hydrophilic backbones which resemble their hydrophilic region. In a preferred embodiment, the graft polymers participating in the three-dimensional hydrogel network all have the same backbone composition, even if they differ in their side chain chirality. For instance, the backbones may represent homopolymer chains of natural or synthetic origin. Alternatively, random or block copolymers can be used which have substantial hydrophilicity even though some of the monomeric units or blocks may not be very hydrophilic. Among the preferred backbones are native and modified or derivatised polysaccharides such as dextran, cellulose including water soluble cellulose ethers such as methyl cellulose, hydroxyethyl cellulose, hydroxypropyl cellulose, hypromellose and carboxymethyl cellulose; pectin, alginate, carrageen, acacia, chitosan, starch, amylose, amylopectin, xanthan, agar-agar, tragacanth, guar gum, karava gum, carob bean gum, etc. A preferred polysaccharide backbone is dextran. Other backbones are polypeptides such as casein, gelatin, collagen, hydrolysed proteins, albumin, ovalbumin, lysozym, poly(lysin), poly(arginine), poly(glutamic acid) or other poly(amino acids). Furthermore, the backbones or major backbone blocks may be selected from poly(vinyl alcohol), poly(ethylene glycol), poly(ethylene oxide), water soluble polyphosphazenes, poly(vinyl pyrrolidone). Another preferred class of backbones or backbone blocks suitable for the invention is that of water soluble (meth)acrylates/(meth)acrylamides, including poly(hydroxyethyl methacrylate), poly(hydroxypropyl methacrylate) and the corresponding acrylamides. A presently preferred acrylic acid-derived backbone is poly(hydroxypropyl methacrylamide) (pHPMAm). In addition to the block or blocks representing hydrophilic region, the backbone may comprise degradable or non-degradable hydrophobic blocks, such as poly(propylene oxide). For example, pluronics, which are block polymers of poly(ethylene oxide) and poly(propylene oxide), may represent suitable backbones.

The molecular weight of the backbone should be selected keeping in mind its specific chemical and physicochemical nature, the requirements in terms of water solubility, the intended degree of substitution with side chains, and other factors. Generally, the molecular weight should be in the range of 1,000 to about 500,000. In most cases, an average molecular weight, and preferably a weight average molecular weight, of about 10,000 to about 150,000 is preferred, as very low molecular weights require a high degree of crosslinking to form a stable hydrogel, whereas very high molecular weights are often difficult to use due to a poorer water solubility and higher viscosity. In order to be able to be excreted by the kidney, it is preferred according to another embodiment that the average molecular weight is not higher than about 80,000 or 100,000, depending on the shape of the molecules.

The backbone polymers are prepared by methods commonly known. Reference is made to WO 00/48576 for a brief description of how to arrive at useful polymers.

In the hydrogels of the invention, the regions participating in the stereocomplex formation and crosslinking are biodegradable. More specifically, they are hydrolysable under physiological conditions. As used herein, hydrolysable under physiological conditions means that at a physiological pH and temperature, they exhibit substantial hydrolytic degradation within time periods of interest for drug delivery or tissue engineering applications, such as over several hours, days, weeks, months, or a few years, without requiring enzymatic catalysis.

The enantiomerically enriched degradable regions of the polymers are preferably oligomeric. An oligomer may be defined as a molecule of intermediate relative molecular mass comprising a small plurality of monomeric units. A molecule or molecular region is regarded as having an intermediate relative molecular mass if it has properties which do vary significantly with the removal of one or a few of the monomeric units. While no absolute limits are generally applicable, oligomers typically comprise a number of monomeric units which is in the region of about 2 to 25.

The enantiomerically enriched degradable regions of the polymers are preferably based on (L)- or (D)-lactate units. In addition, they may contain a relatively low number of non-chiral units which are preferably degradable as well, such as units derived from glycolic acid, caprolactone, or propriolactone.

Oligomeric degradable units can be prepared by generally known methods. In particular, methods of polymerising (D)- or (L)-lactide to prepare oligo(D)- or oligo(L)-lactate are known and e.g. described in De Jong S J, Van Dijk-Wolthuis W N E, Kettenes-van den Bosch J J, Schuyl P J W, and Hennink W E. Monodisperse enatiomeric lactic acid oligomers: preparation, characterization and stereocomplex formation. Macromolecules 31, 6397-6402, 1998. Furthermore, methods for incorporating glycolic acid, caprolactone, or propiolactone units as co-monomers are known.

For example, the degradable regions can be formed by oligomerisation of the respective monomers, which is preferably carried out by using an initiator. The initiator may be incorporated in the oligomer. Such initiators are compounds with a primary or secondary hydroxyl group, e.g.: ethyl lactate or other aliphatic or aromatic lactate esters, benzyl alcohol, lauryl alcohol, 1,4-butanediol, adipic acid, (monomethoxy) PEG, 2-(2-methoxyethoxy) ethanol, or mixtures thereof. Care should be taken that the use of these initiators does not give rise to toxic levels of (reaction products of these initiators in the resulting gel when applied in vivo. For this reason it is preferred to use endogenous compounds or compounds derived from endogenous compounds as an initiator. The use of such compounds as initiator prevents unacceptable (i.e. toxic) levels of these compounds or the reaction products thereof during degradation of the gels. An example of a suitable initiator is ethyl lactate, which is easily hydrolyzed to the relatively harmless compounds ethanol and lactate in e.g. mammals.

When an initiator is used, the resulting oligomers may carry the initiator, or a part of it, as an end group. The amount of initiator relative to the amount of graft monomers can be used to tailor the degree of polymerisation (DP) (see: De Jong S J, Van Dijk-Wolthuis W N E, Kettenes-van den Bosch J J, Schuyl P J W, and Hennink W E. Monodisperse enatiomeric lactic acid oligomers: preparation, characterization and stereocomplex formation. Macromolecules 31, 6397-6402, 1998). Furthermore, the oligomerisation is carried out in the presence of a suitable catalyst. Such a catalyst can, for example, be chosen from stannous octoate, aluminum alkoxides (e.g., aluminum tris (2-propanolate), zinc powder, CaH₂, Sn (IV) tris2-ethylhexanoate, tetraphenylporphinatoaluminum, aluminum triisopropoxide, chiral Schiff s base/aluminum alkoxides, Al (Acac), SALEN-Al-OCH₃, t-BuOLi, Bu₃SnOCH₃, PbO, zinc oxide, diethyl zinc, zinc chloride, stannous chloride, magnesium salt, Zn (Acac) 2, ZnEt₂—Al (OiPr) ₃, (ZnEt₂+AlEt₃+nH₂O), yttrium oxide, or mixtures thereof.

The degree of polymerisation is an important parameter in the design of stereocomplex hydrogels. The degradable regions, whether constituting grafts or backbone blocks, must have a sufficient length in order to enable stereocomplex formation and crosslinking, and to ensure a sufficient gel stability. On the other hand, very long side chains may easily lead to graft polymers which are relatively hydrophobic, i.e. which do not hydrate well to form a hydrogel.

The desirable degree of polymerisation (DP) with regard to the grafts should be determined in consideration of the desired gel properties and the nature of the block or graft polymer that is used. For most lactate blocks, an average DP of at least about 7 is preferred. In order to prepare the hydrogels of the present invention, which are potentially more stable than known stereocomplex hydrogels, it is preferred to select a DP in the range of about 8 to 15, and especially from about 11 to 14.

In the case of graft polymers, the grafts should also have a generally low polydispersity with regard to their chain length. Especially short chains which do not contribute to stereocomplex formation should be excluded (e.g. by chromatographic purification of the oligomers before grafting) if strong and stable hydrogels with long lifetimes are desired. Polydispersity can be expressed by the polydispersity index PDI, which is the ratio of the weight average molecular weight M(w) to the number average molecular weight M(n). Technical polymers typically have polydispersities of 2 or more. In contrast, it is preferred according to the present invention to use relatively monodisperse oligomers as grafts, having a polydispersity of less than about 1.5. More preferably, the grafts have predominantly the same degree of polymerisation, i.e. they are practically monodisperse. Other embodiments relate to grafts selected from lactic acid oligomers with a degree of polymerisation ranging only from 11 to 14, and particularly from 12 to 13. The preparation of graft or block polymers can be effected by mixing the degradable oligomers with or without linking groups and the hydrophilic blocks or backbone polymers in a suitable solvent. Preferably, the grafts are mixed with linking groups. Suitable solvents can be chosen from aprotic solvents, depending on the polymer used, e.g. dimethyl sulfoxide for e.g. dextrans, after which the grafting reaction is carried out under suitable conditions, which conditions can be easily determined by a skilled person. After this the solvent is removed. The degree of substitution can be controlled by changing the amount of (co-) oligomeric graft and water soluble polymer—see De Jong S J, Van Dijk-Wolthuis W N E, Kettenes-van den Bosch J J, Schuyl P J W, and Hennink W E. Monodisperse enatiomeric lactic acid oligomers: preparation, characterization and stereocomplex formation. Macromolecules 31, 6397-6402, 1998.

Another example of synthesising useful graft polymers is described by Lim et al. (Macromol. Rapid Commun. 21, 464-471, 2000). In order to prepare graft polymers having backbones of poly(2-hydroxyethyl methacrylate) and side chains of oligo(D-lactide) or oligo(L-lactide), 2-hydroxyethyl methacrylate (HEMA) was copolymerised with D- or L-lactide in a first step, resulting in an oligomeric D- or L-lactide chain with a terminal HEMA-group. These macromers were in a second step copolymerised with HEMA, leading to the desired graft polymer.

The degradable regions, whether grafts or backbone blocks, are typically attached to the hydrophilic region via linkers. Such linking structure usually represent relatively small chemical groups, but also larger entities such as oligomers could be used. Obviously, the linkers present in the polymers depend on the specific chemistry used for the preparation of the polymers. Most often, linking groups are ester, amide, or urethane groups. In one of the preferred embodiments of the invention, enantiomerically enriched biodegradable side chains are grafted to hydrophilic backbones via ester groups.

Various chemistries are available which can be used to graft side chains on backbones in order to synthesise the graft polymers useful for preparing a hydrogel of the invention. In general, the graft structures can be linked directly to the polymers or by means of a linking group, depending on the reactivity of the groups and the polymer. An example of such a linking group is carbonyldiimidazole (CDI). Such linking groups are converted further when the grafts are linked to the polymer. The linking group could also be applied to enhance the biodegradability of the product. According to one of the embodiments of the present invention, there is a hydrolysable linking group between the water soluble or water dispersible polymer and the oligomeric or co-oligomeric group.

On the other hand, hydrogels with improved stability compared to previously known compositions are more easily achieved when the linking groups have a hydrolytic degradability which does not exceed that of the enantiomerically enriched degradable regions themselves. It is therefore preferred according to the present invention that at least some of the linking groups are hydrolytically more stable than the hydrolysable bonds in the degradable regions. More preferably, practically all linkers are hydrolytically stable relative to the degradable regions. Accordingly, it is preferred that the linking groups is selected from relative stable esters, amides or urethanes. In contrast, hydrolytically labile ester groups, such as carbonate ester groups, should largely be avoided if long-term stability is desired. Of course, for achieving intermediate gel stabilities, it may be useful to incorporate linkers with different degrees of stability.

If the polymers are graft polymers, at least one enantiomerically enriched side chain must be attached to a first graft polymer and at least one enantiomerically enriched side chain having opposite chirality must be attached to a second graft polymer present in the hydrogel in order that stereocomplex formation and crosslinking can occur. In order to form the three-dimensional polymeric network of a hydrogel, most of the graft polymers must have at least two side chains per backbone which are capable of forming crosslinks. More typically, a polymer molecule comprises a much larger number of side chains.

In an alternative embodiment mentioned above, the first and the second graft polymer may optionally be essentially identical, even with regard to the chiral properties of their side chains, provided that each average polymer molecule has at least one side chain of each chiral species. In other words, the hydrogel is in this case composed of only one type of graft polymer which however comprises both types of side chains. According to this embodiment, also intramolecular stereocomplexes can be present in the hydrogel which do not contribute to the crosslinking. Alternatively, the two complementary species of side chains are attached to different backbones, so that the hydrogel is composed of two different graft polymers, a first one only having side chains of one chirality, and a second one only having side chains of the opposite chirality, which is the presently preferred embodiment.

In a more preferred embodiment, however, the hydrogel is based on a mixture of two graft polymers with side chains having opposite chirality. The grafting density, or degree of substitution (DS), should also be selected in consideration of the desired gel strength and stability, the nature and dose of the drug, the length of the side chains, etc. For instance, a very low degree of substitution leads to a low crosslinking density. Consequently, there is for each hydrogel a lower limit of DS which is needed to provide sufficient gel strength and stability. In general, the DS should be in the range of about 1 to 25%. More preferably, it should be selected within the range from about 2 to about 15%. In combination with a DP of about 11 to 14, a DS of about 4 to about 10% is particularly useful.

An important feature of the invention is that the enantiomerically enriched degradable regions participating in stereocomplex formation and crosslinking, or at least the majority of them, have no free terminal hydroxyl groups. In contrast, in most of the known stereocomplex hydrogels, the hydrolysable side chains do have terminal hydroxyl groups. It was surprisingly found by the inventors that the absence of terminal hydroxyl group leads to a much slower hydrolytic degradation of biodegradable oligo- or polyester side chains. Without wishing to be bound by a theory, it is presently believed that the terminal hydroxyl group is involved in one of the mechanisms by which such oligo- or polyester chains hydrolyse.

In particular, it was found by the inventors that stereocomplex hydrogels with side chains prepared from oligomerised hydroxyacids behave very differently depending on whether terminal hydroxyl groups are present or not. For instance, the degradation time of a gel composed of poly(2-hydroxypropyl methacrylamide) (pHPMAm) with side chains of oligo(lactide) with an average DP of about 12 (relating to the side chains) can be increase by a factor of about three by acetylating the terminal hydroxyl groups of the side chains.

As the free terminal hydroxyl group may be capable of becoming involved in the hydrolytic degradation of the side chains, it is important to limit the relative number of such groups in the hydrogel. According to the invention, not all side chains have to be free of terminal hydroxyl group, but at least some of those which are present in the hydrogel. In fact, the degradation time of a hydrogel may be modulated by selecting the ratio of side chains with and without terminal hydroxyl groups. In one of the embodiments, however, all or nearly all side chains are free of terminal hydroxyl group. Thus, gels with a maximum degradation time can be tailored, which are useful for long-time drug release.

In one of the embodiments, the majority (i.e. at least 60%, preferably at least 70%) of the hydrolysable side chains are free of terminal hydroxyl groups. In a further embodiment, practically all, and more preferably all side chains are free of terminal hydroxyl groups.

The oligomerisation of the monomeric units from which the side chains are made, e.g. mostly lactic acid, optionally with some other co-monomers, typically lead to terminal hydroxyl groups. These groups can, in a subsequent step, be reacted with agents such as acetic anhydride. Alternatively, the oligomerisation can be carried out in such a way that the grafts have no free terminal hydroxyl groups, i.e. in which the terminal group is already protected or blocked. A preferred method of avoiding or removing hydroxyl groups is acylation.

Various methods are known by which free hydroxyl group are acylated. For example, acylation can be performed by reacting the hydroxyl group with anhydrides, acyl halides such as acyl chlorides, carboxylic acids or activated carboxylic acids.

Optionally, free hydroxyl groups can also be blocked with any other species which react with alcohols. For example, they can be etherified, silylated, converted into acetals, reacted with isocyanates etc. Methods for carrying out such blocking reactions are generally known in organic chemistry.

Hydrogels can be prepared from the polymers described above in various ways. For instance, they can be produced by combining a first component comprising the first polymer as defined in claim 1 with a second component comprising the second graft polymer in the presence of water. This method can be used when the first and the second graft polymers are chirally different. For instance, the first component may comprise a graft polymer with side chains predominantly composed of oligo(L-lactide), whereas the second component comprises the complementary graft polymer whose side chains contain predominantly oligo(D-lactide). Upon combining the two components—e.g. by mixing—in the presence of water, the graft polymers form crosslinking stereocomplexes, and thus a stereocomplex hydrogel. The water needed for the hydration and swelling of the polymers can be added in the form of a third component, or it may be already present in sufficient amounts in either or both of the first and the second component. In one of the preferred embodiments, the first and the second component are liquid aqueous compositions which are combined by mixing.

If only one type of graft polymer is used in the hydrogel (i.e. when the first and the second graft polymers are identical), the polymer will spontaneously form a hydrogel upon hydration. In this case, a suitable method for preparing the hydrogel may comprise the step of combining a component comprising the graft polymer (e.g. a powder or lyophilisate) with a component comprising the water needed for its hydration. Alternatively, the hydrogel can be prepared from a solution which comprises the polymer, water, and a material preventing the formation of stereocomplexes, such as an organic solvent, a sugar or a salt, by removing this material, or diluting it to such a degree that stereocomplex formation occurs.

Optionally, other excipients may be present in any of the components from which the hydrogel is prepared. The hydrogel composition of the invention, which is a composition comprising a hydrogel as described above, preferably contains other constituents or excipients than only the graft polymer(s) and water. Some or all of these excipients may be already present when the hydrogel is formed. They can be introduced e.g. as constituents of one or both the first and the second component which contain the first and the second graft polymer, or they can be added separately. Alternatively, they can be added to the hydrogel after it has been formed.

A preferred use of the hydrogels, and of compositions based on such gels, is the delivery of pharmaceutically active compounds. As used herein, a pharmaceutically active compound (herein also used interchangeably with “active compound”) is any chemical or biological substance or mixture of substances which is useful for the diagnosis, prevention or treatment of diseases, symptoms, and other conditions of the body, or for influencing a body function. Thus, the hydrogel compositions may comprise one or more of such active compounds. In order to incorporate an active compound, the compound is preferably present when the hydrogel is formed. Alternatively, the hydrogel can be loaded with an active compound, e.g. by soaking the gel in a solution of the compound.

Preferred active compounds are those which are used in chronical or long-term treatment regimen, such as hormones, growth factors, hormone antagonists, antipsychotics, antidepressants, cardiovascular drugs, and the like. In another aspect, a preferred class of active compounds is that of peptides and proteins, in particular proteins, which can be delivered effectively with the hydrogel compositions of the invention, providing drug release over extended time periods, thus eliminating the need for the frequent injection of these compounds which are typically not bioavailable when administered orally.

Among the preferred peptides and proteins are: erythropoetins, such as epoetin alpha, epoetin beta, darbepoetin, haemoglobin raffimer, and analogues or derivatives thereof; interferons, such as interferon alpha, interferon alpha-2b, PEG-interferon alpha-2b, interferon alpha-2a, interferon beta, interferon beta-1a and interferon gamma; insulins; antibodies, such as rituximab, infliximab, trastuzumab, adalimumab, omalizumab, tositumomab, efalizumab, and cetuximab; blood factors such as alteplase, tenecteplase, factor VII(a), factor VIII; colony stimulating factors such as filgrastim, pegfilgrastim; growth hormones such as human growth factor or somatropin; interleukins such as interleukin-2; growth factors such as beclapermin, trafermin, ancetism, keratinocyte growth factor; LHRH analogues such as leuprolide, goserelin, triptorelin, buserelin, nafarelin; vaccines, etanercept, imiglucerase, drotrecogin alpha.

Other preferred active compounds are polysaccharides and oligo- or polynucleotides, antibiotics, and living cells. Optionally, the active compound may be incorporated in the form of a colloidal carrier system such as drug loaded liposomes, polymeric micelles, polymeric nanoparticles, microspheres, poly/lipoplexes, or viral gene delivery vectors.

For drug delivery applications, the hydrogels are preferably used as components of formulations adapted for non-oral administration, such as injectable, implantable, inhalable, or mucosal dosage forms. In order to accommodate a hydrogel in such formulations, the hydrogel itself may be shaped accordingly, e.g. as microparticles (the term being used herein to encompass also microspheres and microcapsules), injectable pellets, single unit dose implants such as rods, sheets, wafers, or other shapes useful for implantation as single units. In one of the presently most preferred embodiments, the hydrogels are shaped as injectable microparticles, having an average diameter selected from about 1 to about 500 μm, and more preferably from about 25 to about 150 μm.

The preparation of such microspheres can be generally performed according to known methods which only need to be adapted to the hydrogels for the invention. For instance, the microspheres can be formed in an emulsion process such as that described in WO 00/48576. Preferably, an emulsion-based method is used which does not require organic solvents.

Injectable or implantable formulations can also be designed so as to gel or solidify in situ. For instance, the graft polymer(s) can be provided in form of an injectable liquid which is prepared from liquid and/or solid premixes shortly prior administration. Since the formation of the stereocomplexes can be adjusted to take sufficiently long so that the mixture can be injected, the gelling occurs in the body. The advantage of this method is that relatively large solid implants can be injected with small needles, possibly without anaesthesia. The premixes, or components, from which the in situ gelling formulation is prepared, can be provided as a kit, which is a package in which the components are contained in individual primary packages.

The hydrogel compositions of the invention and the pharmaceutical formulations may optionally comprise further excipients. These are preferably selected from those excipients which are commonly used in pharmaceutical or food technology. They are primarily used to influence the performance of the formulation, such as the release profile, the viscosity and injectability, or the tolerability. Also, excipients may be used in response to the specific requirements resulting from the nature of the active ingredient, such as stabilisers. Common pharmaceutical excipients which may be useful in hydrogel formulations are humectants, bulking agent, stabilisers, wetting agents, pore forming agents, antioxidants, colouring agents, substances for adjusting the pH and/or the tonicity and the like.

Especially for injectable, implantable and pulmonary administration, the formulations must be sterile. Sterility can be achieved by the selection of appropriate manufacturing processes such as aseptical processing and/or sterilisation of the final product.

For storage, hydrogels may also be dried, and provided in a rewettable form. Especially with this application in mind, one of the embodiments of the invention is represented by a kit from which the hydrogel composition, or a formulation comprising such a composition, can be prepared. Apart from the kit for in situ gelling formulation described above, a pharmaceutical kit may also be designed with two formulation components. For instance, the kit may comprise a first primary package containing a solid-state material, such as granules, a powder, or a lyophilisate, comprising a hydrogel-based composition in a dried state, also referred to as a xerogel; and a second primary package containing a liquid for reconstituting the xerogel to form the hydrogel composition. The liquid comprises water and, optionally, further excipients, such as salts, stabilisers, surfactants etc. The active compound may be present in the xerogel, or in the liquid for reconstitution, or within a third component of the kit. Preferably, however, the active component is incorporated and present in the xerogel, which is preferably shaped as microparticles.

Especially in the form of films, sheets, or gels, the hydrogel compositions of the invention may also be used for tissue engineering applications, or as wound dressings. For these uses, the compositions may or may not comprise a pharmaceutically active compound as defined above. For instance, wound dressings in the form of hydrogel sheets may be useful to cover and protect a wound, which may be sufficient in some instances. In other cases, it may be more useful to incorporate an antimicrobial compound to prevent or treat local infections.

Further embodiments will become obvious from the following examples which illustrate the invention in some of its major aspects, without limiting the scope thereof.

### EXAMPLE 1

### Synthesis of N-(2-hydroxypropyl methacrylamide)-oligo-(L-lactic acid) (HPMAm-oligo-LLA).

A mixture of 10.0 g L-lactide (69.4 mmol), 1.66 g HPMAm (11.6 mmol) and 1.5 mg hydroquinone monomethyl ether (0.012 mmol) was stirred at 120° C. until the lactide was molten, and stannous octoate (0.23 g; 0.58 mmol) was added. The mixture was stirred for 4 h at 130° C. and subsequently cooled to room temperature, to yield HPMAm-oligoLLA.

¹H NMR (CDCl₃): δ (ppm)=5.65 (d, 1H, H^{a}H^{b}C═C), 5.27 (d, 1H, H^{a}H^{b}C═C), 4.90-5.20 (m, C(═O)—CH(—CH₃)—O, CH₂—CH(—O)—CH₃), 4,35 (q, 1H, C(═O)—CH(—CH₃)—OH), 3.6 (m, 1H, CH^{a}H^{b}—CH(—O)—CH₃), 3.25 (m, ¹H, CH^{a}H^{b}—CH(—O)—CH₃), 1.90 (s, 3H, C═C(—CH₃)), 1.35-1.60 (m, C(═O)—CH(—CH₃)—O), 1.20 (d, 3H, CH₂—CH(—O)—CH₃).

Monodisperse HPMAm-oligo(L-lactic acid) was obtained by fractionation, using an ÄKTA purifier (Pharmacia Biotech AB, Sweden) with a preparative HPLC column (Econosphere C8, 10 μm, 250×22 mm; Alltech, Ill., USA). Polydisperse oligomer (1.0 g) was dissolved in 1.5 mL of water/acetonitrile (5/95% w/w) and filtered over a 45 μm filter. 1.5 mL of this solution was injected onto the column. A gradient was run from 50% B (water/acetonitrile 95:5 (w/w)) to 100% B (acetonitrile/water 95:5 (w/w)) in 120 min. The flow rate was 10.0 mL/min; detection by UV (λ=215 nm). The chromatograms were analyzed with Unicorn Analysis module (version 2.30) software. The individual oligomers were collected and fractions with corresponding degrees of polymerization were pooled. The solvent was removed under reduced pressure.

### EXAMPLE 2

### Acetylation of N-(2-hydroxypropyl methacrylamide)-oligo-(L-lactic acid) (HPMAm-oligo-LLA).

The experiment was initially performed as described in example 1. Directly after the ring opening polymerization of L-lactide with HPMAm, the mixture was cooled to 90° C. and a cooler was placed on the reaction flask. 15 mL of acetic anhydride was added and the mixture was stirred for 1 hour. Subsequently, the unreacted acetic anhydride was removed under reduced pressure. The conversion was quantitative according to ¹H NMR.

¹H NMR (CDCl₃): δ (ppm)=5.65 (d, 1H, H^{a}H^{b}C═C), 5.27 (d, 1H, H^{a}H^{b}C═C), 4.90-5.20 (m, C(═O)—CH(—CH₃)—O, CH₂—CH(—O)—CH₃), 3.6 (m, 1H, CH^{a}H^{b}—CH(—O)—CH₃), 3.25 (m, 1H, CH^{a}H^{b}—CH(—O)—CH₃), 2.07 (s, 3H, O—C(═O)—CH₃), 1.90 (s, 3H, C═C(—CH₃)), 1.35-1.60 (m, C(═O)—CH(—CH₃)—O), 1.20 (d, 3H, CH₂—CH(—O)—CH₃).

Monodisperse HPMAm-oligo(L-lactic acid) was obtained by fractionation as described in example 1.

### EXAMPLE 3

### Degradation Studies with Acetylated and Non-Acetylated N-(2-hydroxypropyl methacrylamide)-oligo-(L-lactic acid) (HPMAm-oligo-LLA).

Monodisperse fractions prepared according to examples 1 and 2 were compared with regard to their hydrolytic degradation behaviour. For this experiment, the fractions representing a DP of 7 and of 12 were selected. The degradation experiments were carried out in 20 mL glass bottles, placed in a thermostated water bath at 37° C. The pH was measured before and after degradation at the temperature of the experiment. For the standard degradation experiments 5 mL of stock solution of monodisperse acetylated or non-acetylated HPMAm-oligo(L-lactic acid) in acetonitrile (2 mg/mL) was diluted to a final concentration of 1 mg/mL with 5 mL phosphate buffer (pH 7.2, 100 mM, the ionic strength (μ) adjusted to 0.3 with sodium chloride). The buffer concentrations need to be at least 100 mM to keep the pH at a fixed value. Samples of 400 μL were drawn at regular time intervals and adjusted to pH 4 with 150 μL ammonium acetate buffer (pH 4, 1 M) to inhibit further degradation. The samples were stored at 4° C. prior to analysis with HPLC.

In result, the half life of the 7-mer in acetonitrile/PBS (1:1) was 3.1 hours for the non-acetylated oligomer having a free terminal hydroxyl group, and of 55 hours for the acetylated 7-mer. The 12-mer also showed a half life of 3.1 when in its non-acetylated form, whereas the half life was 35 hours for the acetylated oligomer.

### EXAMPLE 4

### Preparation of Poly(HPMAm) Grafted with Enantiomerically Enriched Oligo(Lactic Acid) Side Chains.

In separate experiments, acetylated and non-acetylated HPMAm-oligo(L- or D-lactic acid) (10.0 g, 9.5 mmol) as prepared in examples 1 an 2, with and without fractionisation, and HPMAm (in varying amounts to achieve varying DS values) were dissolved in 220 mL of freshly distilled dioxane at a temperature of 80° C. Next, AIBN (156 mg, 0.95 mmol) was added. The solution was stirred for 2 hours at 80° C. under a nitrogen atmosphere. The formed graft polymer was precipitated in 1 L of ice-cold diethyl ether. Next, the product was isolated by filtration and dried under vacuum at 40° C., to yield the pHPMA-graft-oligo(lactic acid). The identity of the products was confirmed by ¹H NMR (CDCl₃). Table 1 lists the graft polymers thus obtained.

TABLE 1

| Graft polymer | Chirality | End group | DP (graft) | DS (%) |
|---|---|---|---|---|
| p4-L-Ac | L | —OAcetyl | 12* | 5.2 |
| p4-D-Ac | D | —OAcetyl | 12* | 5.2 |
| p5-L-Ac | L | —OAcetyl | 11.5* | 8.3 |
| p5-D-Ac | D | —OAcetyl | 12* | 8.7 |
| p6-L-Ac | L | —OAcetyl | 11-14 | 5.8 |
| p6-D-Ac | D | —OAcetyl | 11-14 | 5.1 |
| p7-L-H | L | —OH | 12* | 5.3 |
| p7-D-H | D | —OH | 12* | 5.2 |

*Average DP; no fractionisation was performed

### EXAMPLE 5

### Preparation and Characterisation of Stereocomplex Hydrogels from Graft Polymers.

Graft polymer solutions using the polymers obtained according to example 4 were made in acetate buffer (pH 4, 100 mM). Solutions containing equal amounts of L-lactic acid grafted polymer and D-lactic acid grafted polymer of similar DS and DP were mixed and transferred into 2 mL eppendorf tubes, centrifuged (2 min, 13000 rpm) for compression of the material and stored overnight at 4° C. to allow gel-formation. After gelation, the hydrogels were removed from the tubes, cut into a cylindrical shape (length 2 cm, radius 0.46 cm) and weighed accurately (W₀, approx. 1 g). The weighed gels were placed in vials containing 10 mL of phosphate buffer (pH 7.2, 100 mM, ionic strength adjusted to 0.3 with sodium chloride), which were placed in a water bath at 37° C. At regular time intervals, the buffer solutions were completely removed and the weights of the gels (Wₜ) were determined to calculate the swelling ratio. After weighing, new aliquots of buffer were added to the gels. The swelling ratio (Z) is defined as Wₜ/W₀. The hydrogel dissolution time is defined as the time needed for complete degradation (Z=0). Table 2 lists the hydrogels thus obtained, and their characteristics.

TABLE 2

| | | DP | | | Lifetime |
|---|---|---|---|---|---|
| Hydrogel | Polymers | (grafts) | DS (%) | Zₘₐₓ | (d) |
| h4 | p4-L-Ac + p4-D-Ac | 12* | 5.2 | 2.9 | 43.5 |
| h5 | p5-L-Ac + p5-D-Ac | 12* | 8.5 | 3.1 | 84 |
| h6 | p6-L-Ac + p6-D-Ac | 11-14 | 5.5 | 2.6 | 60 |
| h7 | p7-L-H + p7-D-H | 12* | 5.3 | 2.8 | 14.5 |

*Average DP; no fractionisation was performed

The hydrogels h4 and h7 have a comparable graft polymer composition (including DP and DS) except for the terminal groups of the side chains. While the swelling behaviour of the hydrogels in terms of maximum swelling ratio is also comparable, the lifetime of the gel (h4) with terminal acetyl groups is 3 times longer than the life time of gel (h7) with terminal hydroxyl groups. It is believed that the lifetime of the gels is determined by the rate of hydrolytic degradation of the side chains participating in the crosslinking of the polymers.

Hydrogel h5 represents the stereocomplex hydrogel with the longest lifetime found so far, comprising graft polymers without terminal hydroxyl groups.

The comparison of hydrogels h4 and h6, which differ primarily in the degree of polydispersity of the grafts, shows that a low polydispersity can further contribute to expanding the lifetime of a stereocomplex hydrogel according to the invention.

Furthermore, the hydrogels presented in table 2 demonstrate how it is possible to modulate the degradability of stereocomplex hydrogels using the teachings of the invention, potentially leading to tailored compositions for the controlled release of active compounds over periods up to several months.

1. A stereocomplex hydrogel composition, comprising a mixture of a first and a second polymer,
wherein each first and each second polymer has at least one hydrophilic region and at least two oligomeric degradable regions which are hydrolysable under physiological conditions and which comprise enantiomerically enriched chiral monomeric units, and
wherein at least one of the degradable regions of the first polymer and at least one of the degradable regions of the second polymer have predominantly opposite chirality, and
wherein at least some of the degradable regions present in the composition are do not contain free terminal hydroxyl groups.

2. The composition of claim 1, wherein at least one of the first and the second polymer is a graft polymer, of which the hydrophilic region is the backbone and the degradable regions are the side chains.

3. The composition of claim 2, wherein said graft polymer has an average degree of substitution (DS) between about 2% and about 15%.

4. The composition of claim 2, wherein the side chains of said graft polymer have an average degree of polymerisation (DP) in the range of about 7% to 15%.

5. The composition of claim 2, wherein the side chains of said graft polymer have a polydispersity of not more than about 1.5%.

6. The composition of claim 1, wherein at least one of the first and the second polymer is a block polymer comprising three or more blocks, and wherein the degradable regions form at least the terminal blocks of said block polymer.

7. The composition of claim 6, wherein at least one of the first and the second polymer is an ABA block polymer, wherein the hydrophilic region forms the central B block.

8. The composition of claim 1, wherein the degradable region is attached to the hydrophilic region via a linking group selected from the group consisting of ester groups, amide groups, and urethane groups.

9. The composition of claim 8, wherein the linking group is hydrolytically more stable than the degradable region.

10. The composition of claim 1, wherein the hydrophilic region of at least one of the first and the second polymer is derived from a member of the group consisting of
polysaccharides including dextran, starch, cellulose and cellulose derivatives, alginates, pectin, and chitosan;
polypeptides including albumin, lysozyme, poly(amino acids), including poly(lysine) and related copolymers, poly(glutamic acid) and related copolymers;
poly(acrylates)/(acrylamides) including poly(alkyl acrylates)/(alkyl acrylamides) including poly(methacrylate), poly(hydroxyethyl methacrylate), poly(hydroxypropyl methacrylate), poly(hydroxyethyl methacrylamide), poly(hydroxypropyl methacrylamide); and
poly(vinylalcohol), poly(ethylene glycol), water soluble polyphosphazenes, and
mixtures thereof.

11. The composition of claim 1, wherein the degradable regions of at least one of the first and the second polymer are predominantly composed of enantiomerically enriched (L)- and/or (D)-lactate units.

12. The composition of claim 11, wherein at least some of the degradable regions predominantly composed of enantiomerically enriched (L)- and/or (D)-lactate units further comprise monomeric units selected from glycolate, ε-caprolactone, and βpropiolactone units.

13. The composition of claim 1, wherein essentially all degradable regions of the first polymer are of opposite chirality to essentially all degradable regions of the second polymer.

14. The composition of claim 1, wherein at least some of the degradable regions of the first or second polymer bear terminal acyl groups.

15. The composition of claim 1, which is shaped as a plurality of microparticles, as a sheet, or a single implantable unit.

16. The composition of claim 1, further comprising a pharmaceutically active compound.

17. The composition of claim 16, wherein the pharmaceutically active compound is a protein.

18. A method for preparing the composition of claim 1, comprising a step of combining the first polymer and the second polymer in the presence of water and, optionally, other excipients.

19. The method of claim 18, wherein the step of combining the first and the second polymer is conducted in the presence of a pharmaceutically active compound.

20. A kit for the preparation of the composition of claim 1, comprising a first component comprising the first polymer and a second component comprising the second polymer.

21. A kit for the preparation of the composition of claim 1, comprising a first component comprising the first and the second polymer, and a second component comprising water.

22. The kit of claim 21, wherein said first component comprises a xerogel capable of forming a stereocomplex hydrogel upon hydration.

23. The kit of claim 20, further comprising a pharmaceutically active compound.

24. (canceled)

25. The kit of claim 21, further comprising a pharmaceutically active compound.

26. An injectable or implantable pharmaceutical formulation, a wound dressing, or a replacement tissue that comprises the composition of claim 1.
